(19) United States
(12) Patent Application Publication
(10) Pub. No.: US 2025/0249222 A1
(43) Pub. Date: Aug. 7, 2025

(54) CUTTING BALLOON FOLDING SYSTEMS AND METHODS

(71) Applicant: Boston Scientific Scimed, Inc., Maple Grove, MN (US)

(72) Inventors: Gordon John Kocur, Lino Lakes, MN (US); Daniel Ahern, Galway (IE); Conor Ryan, Galway (IE); Conor Robinson, Galway (IE); Michael James Kelly, Galway (IE)

(73) Assignee: Boston Scientific Scimed, Inc., Maple Grove, MN (US)

(21) Appl. No.: 19/046,135

(22) Filed: Feb. 5, 2025

Related U.S. Application Data
(60) Provisional application No. 63/550,892, filed on Feb. 7, 2024.

(51) Int. Cl. A61M 25/10

(52) U.S. Cl. CPC A61M 25/1029; A61M 25/1038; A61M 2025/1004; A61M 2207/10

(57) ABSTRACT

Tools for imparting a plurality of folds to a balloon. An illustrative tool may include a housing defining a central aperture extending along a length of the housing, a plurality of blade pusher elements spaced about a circumference of the housing, and a plurality of balloon pusher elements spaced about a circumference of the housing. The plurality of blade pusher elements may each extende from a radially outward edge to a radially inward portion configured to extend into the central aperture. The plurality of balloon pusher elements may each extend from a radially outward edge to a radially inward end configured to extend into the central aperture. Each balloon pusher element of the plurality of balloon pusher elements may comprise two or more balloon pusher members.

### CROSS REFERENCE TO RELATED APPLICATIONS

The present application claims the benefit of U.S. Provisional Patent Application Ser. No. 63/550,892, filed Feb. 7, 2024, the disclosure of which is incorporated herein by reference.

### TECHNICAL FIELD

The disclosure pertains to methods and systems for folding cutting balloons. More particularly, the disclosure is directed to methods and systems to reduce balloon profile and improve fold uniformity.

### BACKGROUND

Medical balloons are used in the body in a variety of applications including as dilatation devices for compressing plaque and for expanding prosthetic devices such as stents at a desired location in a bodily vessel. Because it is typically necessary for the balloon to traverse a tortuous anatomy as it is being delivered to the desired location in the bodily vessel, it is desirable for the balloon to assume as low a profile as possible. Medical balloons may be folded to reduce the cross-sectional area, diameter, or profile of the balloon and/or to protect the anatomy from blades, injectors, protrusions, atherotomes and/or other surface features. However, some folding systems and methods may result in balloon folds which are not uniform. This may cause variability in profile and/or potential balloon damage during folding which may impact burst pressure, rewrap, and ease of assembly. Of the known medical devices, systems, and methods, each has certain advantages and disadvantages. There is an ongoing need to provide alternative medical devices, systems, and methods including devices, systems, and methods for folding balloons.

### SUMMARY

The disclosure is directed to several alternative designs, materials and methods of manufacturing medical device structures and assemblies.

In a first example, a tool for imparting a plurality of folds to a balloon may comprise a housing defining a central aperture extending along a length of the housing, a plurality of blade pusher elements spaced about a circumference of the housing, the plurality of blade pusher elements each extending from a radially outward edge to a radially inward portion, the radially inward portion configured to extend into the central aperture, and a plurality of balloon pusher elements spaced about a circumference of the housing, the plurality of balloon pusher elements each extending from a radially outward edge to a radially inward end, the radially inward end configured to extend into the central aperture. Each balloon pusher element of the plurality of balloon pusher elements may comprise two or more balloon pusher members.

Alternatively or additionally to any of the examples above, in another example, the plurality of blade pusher elements may be configured to be radially displaced.

Alternatively or additionally to any of the examples above, in another example, the plurality of balloon pusher elements may be configured to be radially displaced.

Alternatively or additionally to any of the examples above, in another example, each balloon pusher member of the two or more balloon pusher members may be independently actuatable.

Alternatively or additionally to any of the examples above, in another example, a radially inward end of at least one of the two or more balloon pusher members may be generally linear.

Alternatively or additionally to any of the examples above, in another example, the radially inward end of the least one of the two or more balloon pusher members may extend generally orthogonal to a longitudinal axis of the at least one balloon pusher member.

Alternatively or additionally to any of the examples above, in another example, the radially inward end of the least one of the two or more balloon pusher members may extend at a non-orthogonal angle to a longitudinal axis of the at least one balloon pusher member.

Alternatively or additionally to any of the examples above, in another example, a radially inward end of at least one of the two or more balloon pusher members may be generally concave.

Alternatively or additionally to any of the examples above, in another example, a radially inward end of at least one of the two or more balloon pusher members may be generally convex.

Alternatively or additionally to any of the examples above, in another example, each balloon pusher element of the plurality of balloon pusher elements may comprise three or more balloon pusher members.

Alternatively or additionally to any of the examples above, in another example, the tool may further comprise a lumen extending through each balloon pusher member of the plurality of balloon pusher members.

Alternatively or additionally to any of the examples above, in another example, the tool may further comprise a plurality of rods circumferentially spaced within the central aperture and extending along a length of the housing.

Alternatively or additionally to any of the examples above, in another example, the plurality of rods may be configured to be rotated about a longitudinal axis of the central aperture in a first direction.

Alternatively or additionally to any of the examples above, in another example, the balloon pusher elements may be configured to be rotated about the longitudinal axis of the central aperture in a second direction opposite the first direction.

Alternatively or additionally to any of the examples above, in another example, the radially inward portion of each blade pusher element of the plurality of pusher elements may define a channel.

In another example, a tool for imparting a plurality of folds to a balloon may comprise a housing defining a central aperture extending along a length of the housing, a plurality of blade pusher elements spaced about a circumference of the housing, the plurality of blade pusher elements each extending from a radially outward edge to a radially inward portion, the radially inward portion configured to extend into the central aperture, a plurality of balloon pusher elements spaced about a circumference of the housing, the plurality of balloon pusher elements each extending from a radially outward edge to a radially inward end, the radially inward end configured to extend into the central aperture, and a plurality of rods circumferentially spaced within the central aperture and extending along a length of the housing.

Alternatively or additionally to any of the examples above, in another example, the plurality of rods may be configured to be rotated about a longitudinal axis of the central aperture in a first direction.

Alternatively or additionally to any of the examples above, in another example, the balloon pusher elements may be configured to be rotated about the longitudinal axis of the central aperture in a second direction opposite the first direction.

Alternatively or additionally to any of the examples above, in another example, the radially inward end of at least one of the plurality of balloon pusher elements may be generally convex.

In another example, a method for folding a balloon may comprise inserting a balloon into a housing of a balloon folding tool, moving a plurality of blade pusher elements radially inwards to compress a first portion of the balloon, and sequentially moving two or more circumferentially adjacent balloon pusher members radially inwards to compress a second portion of the balloon and form a folded portion.

Alternatively or additionally to any of the examples above, in another example, moving the plurality of blade pusher elements radially inwards may be performed at a first pressure.

Alternatively or additionally to any of the examples above, in another example, sequentially moving two or more circumferentially adjacent balloon pusher members radially inwards may be performed at a second pressure less than the first pressure.

Alternatively or additionally to any of the examples above, in another example, after sequentially moving two or more circumferentially adjacent balloon pusher members, the method may further comprise heat setting the balloon.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1 is a schematic cross-sectional side view of an illustrative balloon catheter with cutting or scoring elements that anchor into the vessel wall disposed in a blood vessel;

FIG. 2 is a schematic cross-sectional view of the balloon in a folded or collapsed configuration;

FIG. 3 is a schematic end view of an illustrative balloon folding tool that may be used to fold the balloon into a reduced profile;

FIG. 4 is an enlarged portion of the balloon folding tool of FIG. 3, taken at Detail A of FIG. 3;

FIGS. 5A-5C are schematic partial cross-sectional views of a balloon folding process having an alternative balloon pusher with two independently moving elements per balloon wing that may be used in the balloon folding tool;

FIGS. 6A-6C are schematic partial cross-sectional views of a balloon folding process having an alternative balloon pusher with two independently moving elements per balloon wing with alternative tip shapes that may be used in the balloon folding tool;

FIGS. 7A-7C are schematic partial cross-sectional views of a balloon folding process having an alternative balloon pusher with three independently moving elements per balloon wing that may be used in the balloon folding tool

FIGS. 8A-8C are schematic partial cross-sectional views of a balloon folding process having an alternative balloon pusher with three independently moving elements per balloon wing with alternative tip shapes that may be used in the balloon folding tool;

FIGS. 9A-9D are schematic partial cross-sectional views of a balloon folding process having an alternative balloon pusher with three independently moving elements per balloon wing moved in an alternative sequence that may be used in the balloon folding tool;

FIGS. 10A-10D are schematic partial cross-sectional views of a balloon folding process having an alternative balloon pusher with three independently moving elements per balloon wing moved in an alternative sequence that may be used in the balloon folding tool;

FIG. 11 is an enlarged partial cross-sectional view of the balloon folding tool with the balloon disposed within the central aperture;

FIGS. 12A-12B are schematic partial cross-sectional views of a balloon folding process having an alternative balloon pusher element with a center vacuum or gas pressure orifice that may be used in the balloon folding tool to control the shape of the balloon folds during folding;

FIGS. 13A-13C are schematic partial cross-sectional views of a balloon folding process having an alternative balloon pusher element system that rotates that may be used in the balloon folding tool;

FIG. 14A is a side view of an illustrative balloon catheter with an alignment and loading fixture that may be used with a balloon folding tool;

FIG. 14B is a cross-sectional view of the alignment and loading fixture taken at line 14B-14B of FIG. 14A with a balloon disposed therein;

FIG. 15 is a schematic cross-sectional view of an illustrative balloon protector; and FIG. 16 is a schematic cross-sectional view of the illustrative balloon protector of

FIG. 15 taken at line 16-16 of FIG. 15.

While the invention is amenable to various modifications and alternative forms, specifics thereof have been shown by way of example in the drawings and will be described in detail. It should be understood that however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

Heart and vascular disease are major problems in the United States and throughout the world. Conditions such as atherosclerosis result in blood vessels becoming blocked or narrowed. This blockage can result in lack of oxygenation of the heart, which has significant consequences since the heart muscle must be well oxygenated in order to maintain its blood pumping action, or lack of oxygenation and/or circulation to other regions of the body may occur.

Occluded, stenotic, or narrowed blood vessels, as well as native or synthetic arteriovenous dialysis fistulae, may be treated in a recanalization procedure, such as with an angioplasty balloon catheter advanced over a guidewire to an occlusion so that the balloon is positioned across the occlusion. The balloon is then inflated to enlarge the passageway through the occlusion.

One of the major obstacles in treating coronary artery disease and/or treating blocked blood vessels or fistulae is re-stenosis or re-narrowing of the passageway through the occlusion subsequent to an angioplasty procedure or other recanalization procedure. Evidence has shown that cutting or scoring the stenosis, for example, with an angioplasty balloon equipped with a cutting element, during treatment can reduce incidence of re-stenosis. Additionally, cutting or scoring the stenosis may reduce trauma at the treatment site and/or may reduce the trauma to adjacent healthy tissue. Cutting elements may also be beneficial additions to angioplasty procedures when the targeted de novo occlusion is hardened or calcified. It is believed typical angioplasty balloons, alone, may not be able to expand certain of these hardened lesions. Thus, angioplasty balloons equipped with cutting elements having cutting edges have been developed to attempt to enhance angioplasty treatments.

The folding of medical balloons in order to reduce the cross-sectional area or profile of the balloon is a common industry practice. Balloons may also be folded in order to enhance re-fold characteristics of the balloon during deflation. The folding of cutting and scoring balloons may have unique requirements and limitations to account for the presence of cutting and scoring elements on and/or over the balloon and more specifically to the location of the cutting and/or scoring element with balloon folds. Devices used for imparting the desired folded configuration vary. Some devices fold the balloon by imparting an inward radial force about the periphery of the balloon using a plurality of rigid blades and/or jaws which are distributed about the periphery of the balloon. However, there may be variability in the shape of the folding balloon wings which can lead to other variabilities, such as, but not limited to, variabilities in the crossing profile, variabilities in how the balloon protector fits, balloon migration to the blade channel in the balloon protector, balloon damage, variabilities in balloon rewrap, and the like. Described herein are methods and systems for folding balloons with improvements in the fold shape of the balloon with reduced variability.

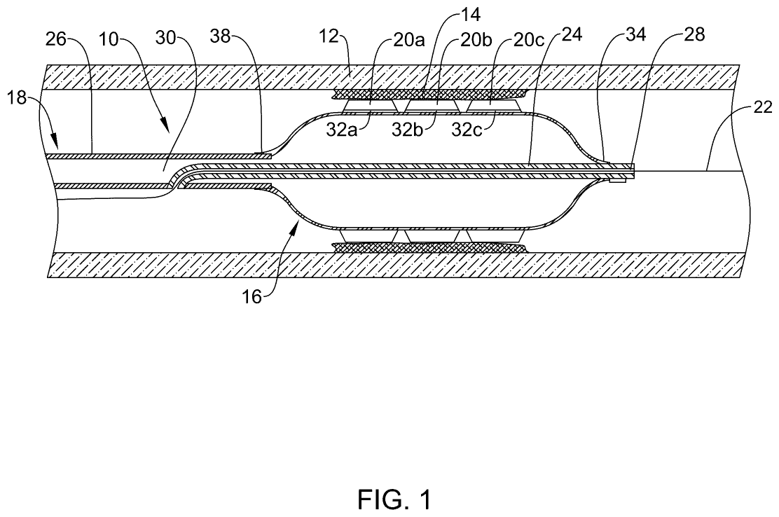

FIG. 1 is a partial cross-sectional side view of an illustrative catheter 10 disposed in a blood vessel 12 and positioned adjacent to an intravascular lesion 14. The catheter 10 may include a balloon 16 coupled to a catheter shaft 18. One or more cutting members or blades 20a, 20b, 20c (collectively, 20) may be mounted on the balloon 16. In some cases, the one or more cutting members 20 may be mounted on a mounting pad 32a, 32b, 32c (collectively, 32) which, in turn, may be coupled to the balloon 16. For brevity, not all of the cutting blades 20 or mounting pads 32 have been identified with a reference number. In general, the catheter 10 may be advanced over a guidewire 22, through the vasculature, to a target area. Once positioned at the target location in the vasculature, the balloon 16 can be inflated to exert a radially outward force on the lesion 14, as the cutting members 20 engage the lesion 14. Thus, the cutting members 20 may cut or score the lesion 14 to facilitate enlarging the lumen proximate the lesion 14. The target area may be within any suitable peripheral or cardiac vessel lumen location.

The balloon 16 may have a length in the range of about 6 to 150 millimeters (mm) or about 6 to 20 mm for coronary applications. In some instances, the balloon 16 may have an outer diameter in the range of about 2 to 12 mm, about 2 to 5 mm for coronary applications. However, the length and/or outer diameter of the balloon 16 may vary based on the application.

The cutting members 20 may vary in number, position, and arrangement about the balloon 16. For example, the catheter 10 may include one, two, three, four, five, six, or more cutting members 20 that are disposed at any position along the balloon 16 and in a regular, irregular, or any other suitable pattern. For example, in some embodiments the balloon 16 may include a plurality of cutting members 20 longitudinally arranged symmetrically around the circumference of the balloon 16.

The cutting members 20 may be made from any suitable material such as a metal, metal alloy, polymer, metal-polymer composite, and the like, or any other suitable material. For example, cutting members 20 may be made from stainless steel, titanium, nickel-titanium alloys, tantalum, iron-cobalt-nickel alloys, or other metallic materials in some instances.

The balloon 16 may be made from typical angioplasty balloon materials including polymers such as polyethylene terephthalate (PET), polytetrafluoroethylene (PTFE), ethylene tetrafluoroethylene (ETFE), polybutylene terephthalate (PBT), polyurethane, polyvinylchloride (PVC), polyether-ester, polyester, polyamide, elastomeric polyamides, polyether block amide (PEBA), as well as other suitable materials, or mixtures, combinations, copolymers thereof, polymer/metal composites, and the like. In some instances, the balloon 16 may include a single layer of material, whereas in other instances the balloon 16 may be of a multi-layer construction, including a plurality of layers of materials. For instance, the balloon 16 may be formed as a co-extrusion or tri-layer extrusion in some instances.

The shaft 18 may be a catheter shaft, similar to typical catheter shafts. For example, the catheter shaft 18 may include an outer tubular member 26 and an inner tubular member 24 extending through at least a portion of the outer tubular member 26. The tubular members 24, 26 may be manufactured from a number of different materials. For example, the tubular members 24, 26 may be made of metals, metal alloys, polymers, metal-polymer composites or any other suitable materials.

The tubular members 24, 26 may be arranged in any appropriate way. For example, in some embodiments the inner tubular member 24 can be disposed coaxially within the outer tubular member 26. According to these embodiments, the inner and outer tubular members 24, 26 may or may not be secured to one another along the general longitudinal axis of the catheter shaft 18. Alternatively, the inner tubular member 24 may follow the inner wall or otherwise be disposed adjacent the inner wall of the outer tubular member 26. In other embodiments, the tubular members 24, 26 may be arranged in another desired fashion.

The inner tubular member 24 may include an inner lumen 28. In at least some embodiments, the inner lumen 28 is a guidewire lumen for receiving the guidewire 22 therethrough. Accordingly, the catheter 10 can be advanced over the guidewire 22 to the desired location. The guidewire lumen 28 may extend along essentially the entire length of the catheter shaft 18 such that catheter 10 resembles traditional “over-the-wire” catheters. Alternatively, the guidewire lumen 28 may extend along only a portion of the catheter shaft 18 such that the catheter 10 resembles “single-operator-exchange” or “rapid-exchange” catheters.

The catheter shaft 18 may also include an inflation lumen 30 that may be used, for example, to transport inflation media to and from the balloon 16 to selectively inflate and/or deflate the balloon 16. The location and position of the inflation lumen 30 may vary, depending on the configuration of the tubular members 24, 26. For example, when the outer tubular member 26 surrounds the inner tubular member 24, the inflation lumen 30 may be defined within the space between the tubular members 24, 26. In embodiments in which the outer tubular member 26 is disposed alongside the inner tubular member 24, then the inflation lumen 30 may be the lumen of the outer tubular member 26.

The balloon 16 may be coupled to the catheter shaft 18 in any of a number of suitable ways. For example, the balloon 16 may be adhesively or thermally bonded to the catheter shaft 18. In some embodiments, a proximal waist 38 of the balloon 16 may be bonded to the catheter shaft 18, for example, bonded to the distal end of the outer tubular member 26, and a distal waist 34 of the balloon 16 may be bonded to the catheter shaft 18, for example, bonded to the distal end of the inner tubular member 24. The exact bonding positions, however, may vary.

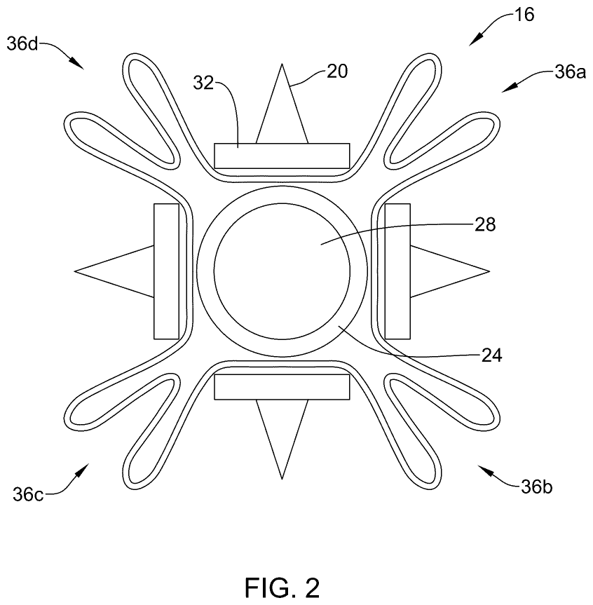

FIG. 2 is a schematic cross-sectional view of the balloon 16 in a folded or collapsed configuration. A balloon folding tool may be used to fold the balloon 16 to reduce the cross-sectional dimension thereof for delivery. The balloon folding tool may compress the balloon 16 such that the balloon 16 includes one or more “wings” or wing-shaped regions 36a, 36b, 36c, 36d (collectively, 36) when the balloon 16 is deflated. In some instances, the wings 36 may be configured so that the cutting members 20 (e.g., 20a, 20b, 20c) can be positioned at the inward-most positions of the deflated balloon 16, with the wings of the balloon folds positioned between adjacent cutting members 20. For brevity, not all of the cutting blades 20 or mounting pads 32 have been identified with a reference number. This arrangement may reduce the exposure of the cutting members 20 to the folded balloon wings 36 and the blood vessel during delivery of the balloon 16 to the lesion 14.

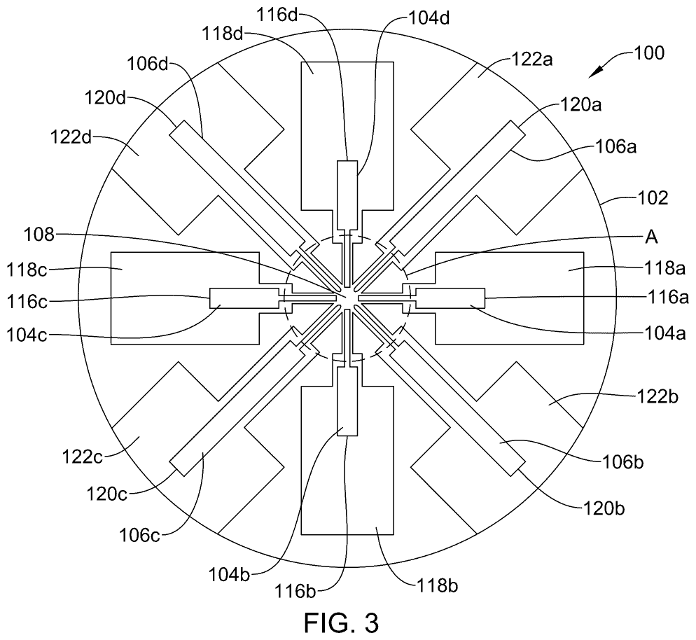

FIG. 3 is a schematic end view of an illustrative balloon folding tool 100 that may be used to fold the balloon 16 into a reduced profile. The balloon folding tool 100 may including a housing 102, a first plurality of blade pusher elements 104a, 104b, 104c, 104d (collectively, 104), and a second plurality of balloon pusher elements 106a, 106b, 106c, 106d (collectively, 104). The housing 102 may extend from a first end to a second end with a length extending therebetween. The length of the housing 102 may be sized such that an entirety of the cutting balloon 16 may be positioned within housing 102.

While the illustrative balloon folding tool 100 is illustrated as including four blade pusher elements 104 and four balloon pusher elements 106, it is contemplated that there may be fewer than four or more than four blade pusher elements 104 and there may be fewer than four or more than four balloon pusher elements 106. It is contemplated that the number of pusher elements in each plurality of pusher elements 104, 106 may depend, at least in part on the number of circumferential locations of the cutting blades 20. In some cases, the balloon folding tool 100 may include a same number of blade pusher elements 104 and balloon pusher elements 106. However, this is not required. In some embodiments, the balloon folding tool 100 may include differing numbers of blade pusher elements 104 and balloon pusher elements 106, as desired.

The blade pusher elements 104 and the balloon pusher elements 106 may be circumferentially spaced about the circumference of the housing 102 and about a central aperture 108 extending through a central portion of the housing 102. The central aperture 108 may be sized and shaped to receive an inflated cutting balloon 16 therethrough. Generally, the blade pusher elements 104 may be configured to engage and radially displace a portion of the balloon 16 adjacent to or at the cutting blade 20. The blade pusher elements 104 may be circumferentially spaced about the central aperture 108 in a manner to align with the cutting blades 20. For example, when the balloon 16 includes four longitudinally extending cutting blades 20 (or cutting blade arrays) that are evenly spaced about the circumference of the balloon 16, the blade pusher elements 104 may be spaced approximately 90° from one another. When the balloon 16 includes three longitudinally extending cutting blades 20 (or cutting blade arrays) that are evenly spaced about the circumference of the balloon 16, the first plurality of pusher elements 104 may be spaced approximately 120° from one another. At least one balloon pusher element 106 may be positioned between each adjacent pair of blade pusher elements 104.

The blade pusher elements 104 and the balloon pusher elements 106 may have a length similar to a length of the housing 102. For example, the blade pusher elements 104 and the balloon pusher elements 106 may have length similar to a length of the balloon 16 such that, when actuated, the blade pusher elements 104 and the balloon pusher elements 106 are configured to exert a radially inward force along an entire length of the balloon 16. However, this is not required. In some examples, the blade pusher elements 104 and the balloon pusher elements 106 may have a length that is less than a length of the housing 102 or less than a length of the balloon 16. In some cases, more than one blade pusher element 104 or more than one balloon pusher element 106 may be provided along the length of the balloon 16 for a particular circumferential location. It is contemplated that the blade pusher elements 104 and the balloon pusher elements 106 may be arranged in any manner desired along the length and/or circumference of the housing 102.

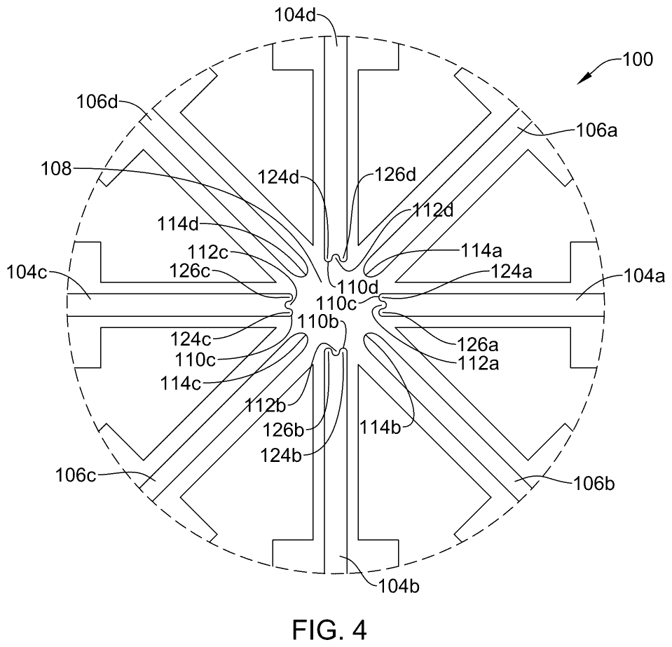

The blade pusher elements 104 may extend from a radially outward edge 116a, 116b, 116c, 116d (collectively, 116) to a radially inward portion 110a, 110b, 110c, 110d (collectively, 110) (see, for example, FIG. 4). The blade pusher elements 104 may be movably disposed within a respective channel 118a, 118b, 118c, 118d (collectively, 118). The channels 118 may be sized and shaped to allow for radially inward and outward movement of the blade pusher elements 104. The channels 118 may extend along the length of the housing 102. While not explicitly shown, the balloon folding tool 100 may further include one or more actuation mechanisms movably coupled to the blade pusher elements 104. The actuation mechanisms may be configured to radially displace the blade pusher elements 104 to a specific location, a force, or both.

The balloon pusher elements 106 may extend from a radially outward edge 120a, 120b, 120c, 120d (collectively, 120) to a radially inward portion 114a, 114b, 114c, 114d (collectively, 114) (see, for example, FIG. 4). The balloon pusher elements 106 may be movably disposed within a respective channel 122a, 122b, 122c, 122d (collectively, 122). The channels 122 may be sized and shaped to allow for radially inward and outward movement of the balloon pusher elements 106. The channels 122 may extend along the length of the housing 102. While not explicitly shown, the balloon folding tool 100 may further include one or more actuation mechanisms movably coupled to the balloon pusher elements 106. The actuation mechanisms may be configured to radially displace the balloon pusher elements 106 to a specific location, a force, or both.

Referring additionally to FIG. 4, which shows an enlarged portion of the balloon folding tool 100 taken at Detail A of FIG. 3, the radially inward portion 110 of each of blade pusher element 104 may be generally “U” shaped having a first leg 124a, 124b, 124c, 124d (collectively, 124) and a second leg 126a, 126b, 126c, 126d (collectively, 126) and defining a channel 112a, 112b, 112c, 112d (collectively, 112) therebetween. The channel 112 may be sized and shaped to receive the cutting blade 20 therein. The free ends of the generally “U” shaped radially inward portion 110 may be configured to be positioned on either side of the blade 20. In some cases, the free ends generally “U” shaped radially inward portion 110 may be configured to engage the mounting pad 32. The radially inward portion 114 of each balloon pusher element 106 may be curved. However, other atraumatic shapes may be used as desired.

To fold the balloon 16, the balloon 16 may be inserted into the central aperture 108 of the balloon folding tool 100 with the balloon 16 in an inflated configuration. In some examples, a funnel or guide device may be used to facilitate insertion of the balloon 16 into the balloon folding tool 100 while aligning the cutting blades 20 with the blade pusher elements 104. Once the balloon 16 is within the central aperture 108, the blade pusher elements 104 may be advanced radially inwards towards a center of the central aperture 108. The cutting blades 20 may be received within the channels 112 and moved radially inwards along with the blade pusher elements 104. Radial movement of the blade pusher elements 104 may stop at a specific location, force, or when the region of the balloon adjacent to each of the mounting pads 32 contacts or nearly contacts the outer surface of the inner tubular member 24. Next, the balloon pusher elements 106 may be advanced radially inwards towards a center of the central aperture 108. It is contemplated that the balloon pusher elements 106 may be advanced with the balloon 16 under a lower pressure than while the blade pusher elements 104 are advanced. However, this is not required. The blade pusher elements 104 may remain in the radially inwards position as the balloon pusher elements 106 are advanced to maintain the mounting pads 32 in the most radially inward position. The balloon pusher elements 106 may radially compress the region of the balloon 16 between the cutting blades 20 to form wings 36, as shown in FIG. 2. The wings 36 may have a generally “Y” or “V” shape. However, the wings 36 may not be symmetrical. Further, the wings 36 may vary in shape from one to another. In some cases, the arms of the “Y” or “V” shape may fold on themselves randomly leading to variability in the balloon 16. The housing 102 may be heated to heat set the balloon 16 in the folded configuration. Heat setting may occur with the blade pusher elements 104 and the balloon pusher elements 106 in the radially advanced configuration. The blade pusher elements 104 and the balloon pusher elements 106 may be moved radially outward or radially withdrawn and the balloon 16 removed from the balloon folding tool 100.

It is contemplated that the blade pusher elements 104 and the balloon pusher elements 106 may be actuated in a number of different sequences. For example, in some embodiments, the blade pusher elements 104 and the balloon pusher elements 106 may be actuated substantially simultaneously at similar advancement rates. In other examples, the blade pusher elements 104 and the balloon pusher elements 106 may be actuated substantially simultaneously with differing rates of advancement. In yet other examples, the blade pusher elements 104 may be actuated prior to actuating the balloon pusher elements 106. Further, all of the blade pusher elements 104 need not be actuated simultaneously. In some cases, one or more of the blade pusher elements 104 may be actuated sequentially at the same rate or at different rates. Similarly, all of the balloon pusher elements 106 need not be actuated simultaneously. In some cases, one or more of the balloon pusher elements 106 may be actuated sequentially at the same rate or at different rates. These are just some examples. It is contemplated that the blade pusher elements 104 and/or balloon pusher elements 106 may be actuated (advanced and/or retracted) at rates from about 0.3 millimeters (mm) per second (sec) to about 4 mm/sec. In one illustrative example, the blade pusher elements 104 may be advanced at a rate of above 3 mm/sec and the balloon pusher elements 106 may be advanced at a rate of 0.5 mm/sec. Once the cycle is complete, the blade pusher elements 104 and/or balloon pusher elements 106 may be retracted at a rate of about 3 mm/sec. This is just one example. The blade pusher elements 104 and/or balloon pusher elements 106 may be actuated in any sequence and/or at any rate desired.

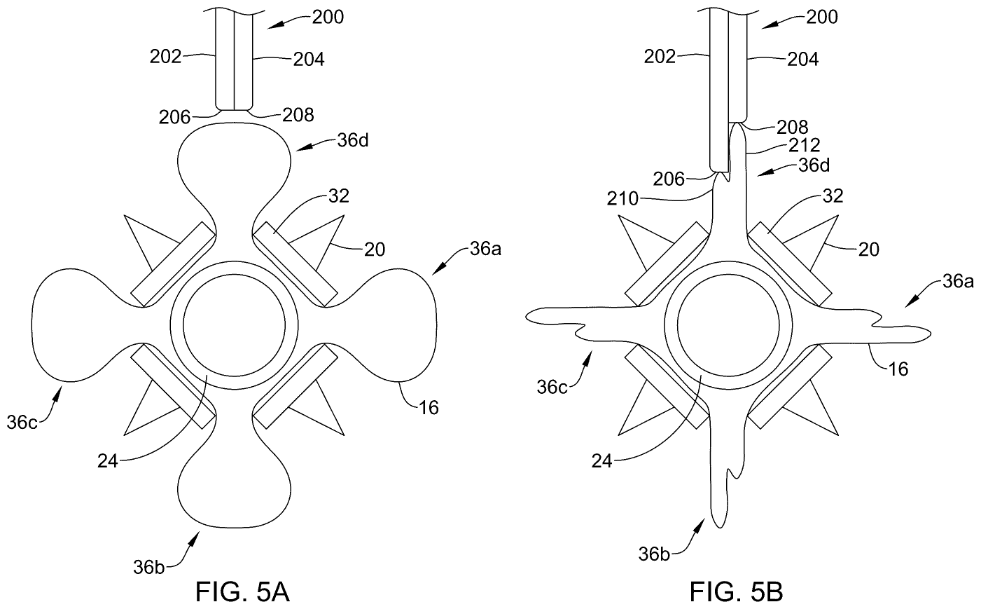

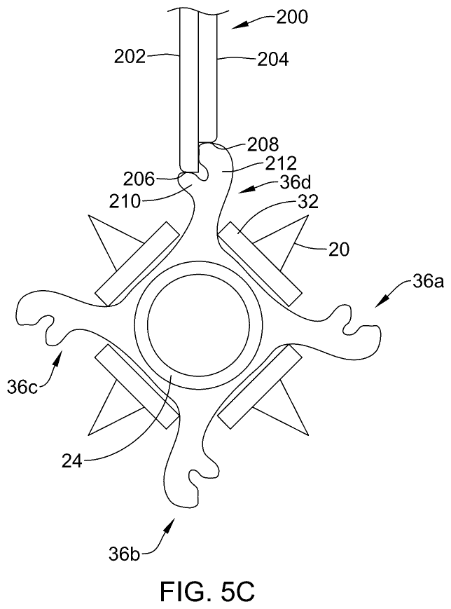

FIGS. 5A-5C are schematic partial cross-sectional views of a balloon folding process having an alternative balloon pusher element 200 that may be used in the balloon folding tool 100. While a single balloon pusher element 200 is shown in FIGS. 5A-5C, it should be understood that a plurality of balloon pusher elements 200 may be utilized. For example, a balloon pusher element 200 may be provided for each wing 36. Further, the balloon pusher elements 200 may each be actuated in a similar manner to form wings 36 having similar shapes. The balloon pusher element 200 may be used in the balloon folding tool 100 in place of one or more of the balloon pusher elements 106.

The balloon pusher element 200 may include a first balloon pusher member 202 and a second balloon pusher member 204. The first and second balloon pusher members 202, 204 may be individually actuatable. For example, the first balloon pusher member 202 may be movable independent of the second balloon pusher member 204 and the second balloon pusher member 204 may be movable independent of the first balloon pusher member 202. Each of the first and second balloon pusher members 202, 204 extend from a radially outward end (not explicitly shown) to a radially inward end 206, 208. The radially inward ends 206, 208 may be generally flat or linear and extend at a generally orthogonal angle relative to the longitudinal axis of the first and second balloon pusher members 202, 204. The radially inward ends 206, 208 may extend at non-orthogonal angles, as desired. The radially inward ends 206, 208 may take other shapes. Other shapes of the radially inward ends 206, 208 may include, but are not limited to, pear shaped, rounded, concave, convex, undulating, or the like. It is contemplated that the shape of the radially inward ends 206, 208 may impact the final shape of the wings 36. While the radially inward ends 206, 208 are shown and described as being of the same shape, the radially inward ends 206, 208 may have differing shapes, as desired. It is further contemplated that the first and second balloon pusher members 202, 204 may have similar widths or differing widths, as desired.

In FIGS. 5A-5C, the portions of the balloon 16 adjacent to the blades 20 have already been folded. For brevity and case of understanding, the blade pusher elements (e.g., similar in form and function to blade pusher elements 104 described herein) are not shown. However, it should be understood that the balloon pusher elements 200 may be used in combination with blade pusher elements. FIG. 5A illustrates the balloon folding process just after the blade pusher elements have been actuated. In FIG. 5B, the first balloon pusher member 202 of the balloon pusher element 200 has been advanced radially inwards while the second balloon pusher member 204 remains stationary. The first balloon pusher member 202 may push or displace a first portion 210 of the balloon 16 adjacent thereto to reduce the outer profile of the balloon 16. Next, as shown in FIG. 5C, the second balloon pusher member 204 may push or displace a second portion 212 of the balloon 16 adjacent thereto to reduce the outer profile of another portion of the balloon 16. In some examples, the second balloon pusher member 204 may be actuated while the first balloon pusher member 202 remains in an advanced radially inwards configuration (e.g., to hold the first portion of the balloon 16 in the folded configuration). In other examples, the first balloon pusher member 202 may be partially or fully radially retracted (e.g., moved radially outwards) before the second balloon pusher member 204 is moved radially inwards or while the second balloon pusher member 204 is being moved radially inwards. In some examples, the second balloon pusher member 204 may fold the second portion 212 of the balloon wing 36d over the first portion 210 (folded by the first balloon pusher member 202) to form a “C” shaped wing 36. However, it is contemplated that the order or sequence of actuation of the first and second balloon pusher members 202, 204 may be manipulated to change a shape of the wing 36.

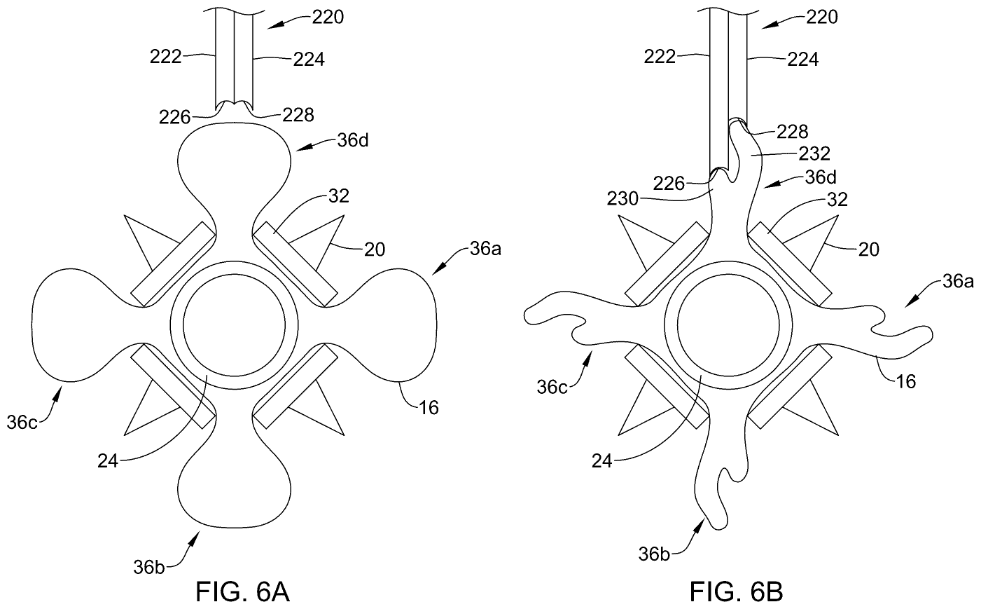

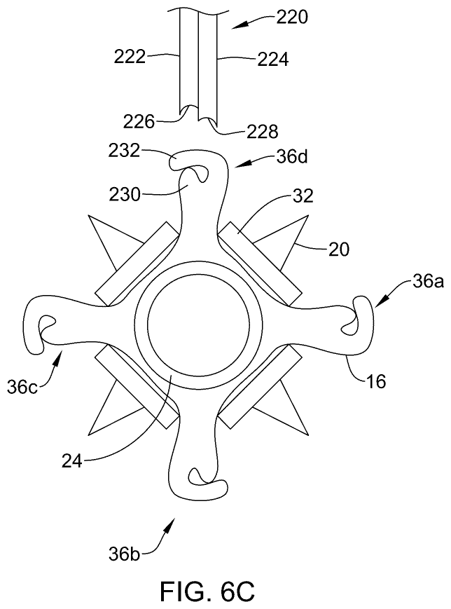

FIGS. 6A-6C are schematic partial cross-sectional views of a balloon folding process having an alternative balloon pusher element 220 that may be used in the balloon folding tool 100. While a single balloon pusher element 220 is shown in FIGS. 6A-6C, it should be understood that a plurality of balloon pusher elements 220 may be utilized. For example, a balloon pusher element 220 may be provided for each wing 36. Further, the balloon pusher elements 220 may each be actuated in a similar manner to form wings 36 having similar shapes. The balloon pusher element 220 may be used in the balloon folding tool 100 in place of one or more of the balloon pusher elements 106.

The balloon pusher element 220 may include a first balloon pusher member 222 and a second balloon pusher member 224. The first and second balloon pusher members 222, 224 may be individually actuatable. For example, the first balloon pusher member 222 may be movable independent of the second balloon pusher member 224 and the second balloon pusher member 224 may be movable independent of the first balloon pusher member 222. Each of the first and second balloon pusher members 222, 224 extend from a radially outward end (not explicitly shown) to a radially inward end 226, 228. The radially inward ends 226, 228 may be generally concave. In some cases, the radially inward ends 226, 228 may be mirror images of one another. In some embodiments, an outer edge of each of the first and second balloon pusher members 222, 224 may be longer than an inner edge. However, this is not required. The radially inward ends 226, 228 may take other shapes and/or configurations as desired. Other shapes of the radially inward ends 226, 228 may include, but are not limited to, pear shaped, rounded, flat (extending at orthogonal or non-orthogonal angles to a longitudinal axis of the first and second balloon pusher members 222, 224), convex, undulating, or the like. It is contemplated that the shape of the radially inward ends 226, 228 may impact the final shape of the wings 36. While the radially inward ends 226, 228 are shown and described as being of a similar shape, the radially inward ends 226, 228 may have differing shapes, as desired. It is further contemplated that the first and second balloon pusher members 222, 224 may have similar widths or differing widths, as desired.

In FIGS. 6A-6C, the portions of the balloon 16 adjacent to the blades 20 have already been folded. For brevity and case of understanding, the blade pusher elements (e.g., similar in form and function to blade pusher elements 104 described herein) are not shown. However, it should be understood that the balloon pusher elements 220 may be used in combination with blade pusher elements. FIG. 6A illustrates the balloon folding process just after the blade pusher elements have been actuated. In FIG. 6B, the first balloon pusher member 222 of the balloon pusher element 220 has been advanced radially inwards while the second balloon pusher member 224 remains stationary. The first balloon pusher member 222 may push or displace a first portion 230 of the balloon 16 adjacent thereto to reduce the outer profile of the balloon 16. Next, as shown in FIG. 6C, the second balloon pusher member 224 may push or displace a second portion 232 of the balloon 16 adjacent thereto to reduce the outer profile of a second portion 232 of the balloon 16. In some examples, the second balloon pusher member 224 may be actuated while the first balloon pusher member 222 remains in an advanced radially inwards configuration (e.g., to hold the first portion of the balloon 16 in the folded configuration). In other examples, the first balloon pusher member 222 may be partially or fully radially retracted (e.g., moved radially outwards) before the second balloon pusher member 224 is moved radially inwards or while the second balloon pusher member 224 is being moved radially inwards. In some examples, the second balloon pusher member 224 may fold the second portion 232 of the balloon wing 36d over the first portion 230 (folded by the first balloon pusher member 222) to form a wing 36 having a Y-C hybrid shape. Said differently, the second portion 232 of the balloon wing 36d may fold over the first portion 230 while a “V” shaped gap remains between lower portions of the first balloon portion 230 and the second balloon portion 232. However, it is contemplated that the order or sequence of actuation of the first and second balloon pusher members 222, 224 may be manipulated to change a shape of the wing 36.

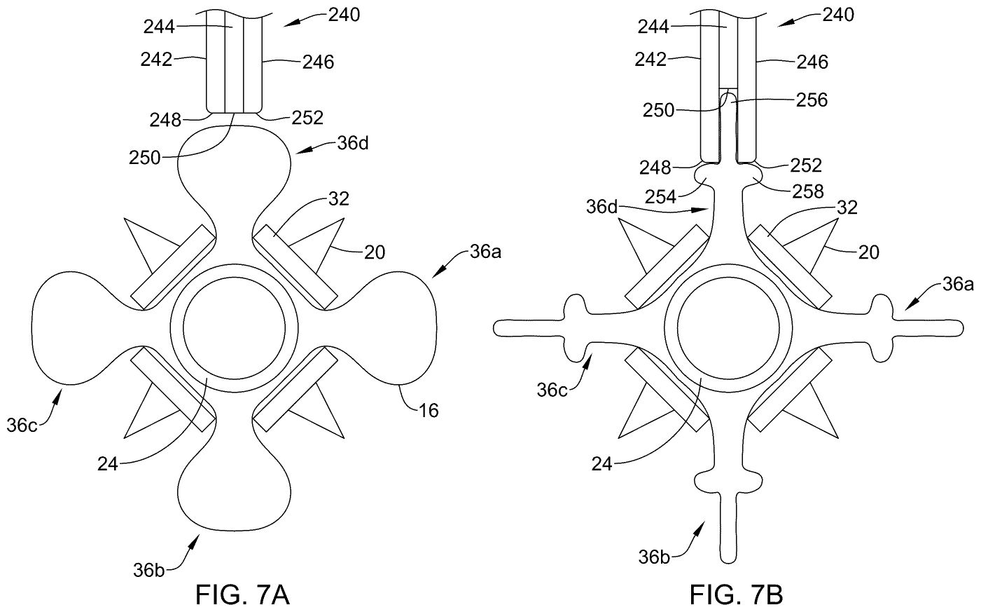

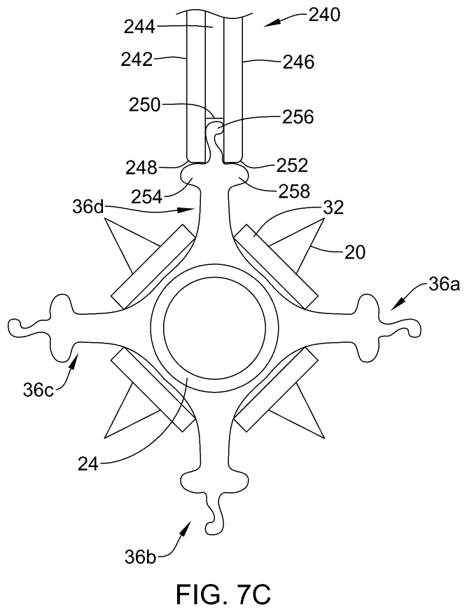

FIGS. 7A-7C are schematic partial cross-sectional views of a balloon folding process having an alternative balloon pusher element 240 that may be used in the balloon folding tool 100. While a single balloon pusher element 240 is shown in FIGS. 7A-7C, it should be understood that a plurality of balloon pusher elements 240 may be utilized. For example, a balloon pusher element 240 may be provided for each wing 36. Further, the balloon pusher elements 240 may each be actuated in a similar manner to form wings 36 having similar shapes. The balloon pusher element 240 may be used in the balloon folding tool 100 in place of one or more of the balloon pusher elements 106.

The balloon pusher element 240 may include a first balloon pusher member 242, a second balloon pusher member 244, and a third balloon pusher member 246. The first, second, and third balloon pusher members 242, 244, 246 may be individually actuatable. For example, the first balloon pusher member 242 may be movable independent of the second balloon pusher member 244 and the third balloon pusher member 246, the second balloon pusher member 244 may be movable independent of the first balloon pusher member 242 and the third balloon pusher member 246, and the third balloon pusher member 248 may be movable independent of the first balloon pusher member 242 and the second balloon pusher member 244. Each of the first, second, and third balloon pusher members 242, 244, 246 extend from a radially outward end (not explicitly shown) to a radially inward end 248, 250, 252. The radially inward ends 248, 250, 252 may be generally flat or linear. In some cases, one or more of the radially inwards ends 248, 250, 252 may extend at a generally orthogonal angle to a longitudinal axis of the balloon pusher members 242, 244, 246. In other examples, one or more of the radially inwards ends 248, 250, 252 may extend at a non-orthogonal angle to a longitudinal axis of the balloon pusher members 242, 244, 246. However, this is not required. The radially inward ends 248, 250, 252 may take other shapes and/or configurations as desired. Other shapes of the radially inward ends 248, 250, 252 may include, but are not limited to, pear shaped, rounded, concave, convex, undulating, or the like. It is contemplated that the shape of the radially inward ends 248, 250, 252 may impact the final shape of the wings 36. While the radially inward ends 248, 250, 252 are shown and described as being of a similar shape, the radially inward ends 248, 250, 252 may have differing shapes, as desired. It is further contemplated that the first, second, and third balloon pusher members 242, 244, 246 may have similar widths or differing widths, as desired.

In FIGS. 7A-7C, the portions of the balloon 16 adjacent to the blades 20 have already been folded. For brevity and case of understanding, the blade pusher elements (e.g., similar in form and function to blade pusher elements 104 described herein) are not shown. However, it should be understood that the balloon pusher elements 240 may be used in combination with blade pusher elements. FIG. 7A illustrates the balloon folding process just after the blade pusher elements have been actuated. In FIG. 7B, the first balloon pusher member 242 and the third balloon pusher member 246 of the balloon pusher element 240 have been advanced radially inwards while the second balloon pusher member 244 remains stationary. The first balloon pusher member 242 may push or displace a first portion 254 of the balloon 16 adjacent thereto to reduce the outer profile of the balloon 16 and the third balloon pusher member 246 may push or displace a third portion 258 of the balloon 16 adjacent thereto to reduce the outer profile of the balloon 16. In some examples, the first and third balloon portions 254, 258 may extend generally orthogonal to the second balloon portion 256. However, this is not required. Next, as shown in FIG. 7C, the second balloon pusher member 244 may push or displace a second portion 256 of the balloon 16 adjacent thereto to reduce the outer profile of a second portion 256 of the balloon 16. In some examples, the second balloon pusher member 244 may be actuated while the first and third balloon pusher members 242, 246 remain in an advanced radially inwards configuration (e.g., to hold the first portion 254 and third portion 258 of the balloon 16 in the folded configuration). In other examples, the first balloon pusher member 242 and/or the third balloon pusher member 246 may be partially or fully radially retracted (e.g., moved radially outwards) before the second balloon pusher member 244 is moved radially inwards or while the second balloon pusher member 244 is being moved radially inwards. In some examples, the second balloon pusher member 244 may fold the second portion 256 on itself to form a wing 36 having a curved or undulating central portion 256 and laterally extending side portions 254, 258. However, it is contemplated that the order or sequence of actuation of the first, second, and third balloon pusher members 242, 244, 246 may be manipulated to change a shape of the wing 36.

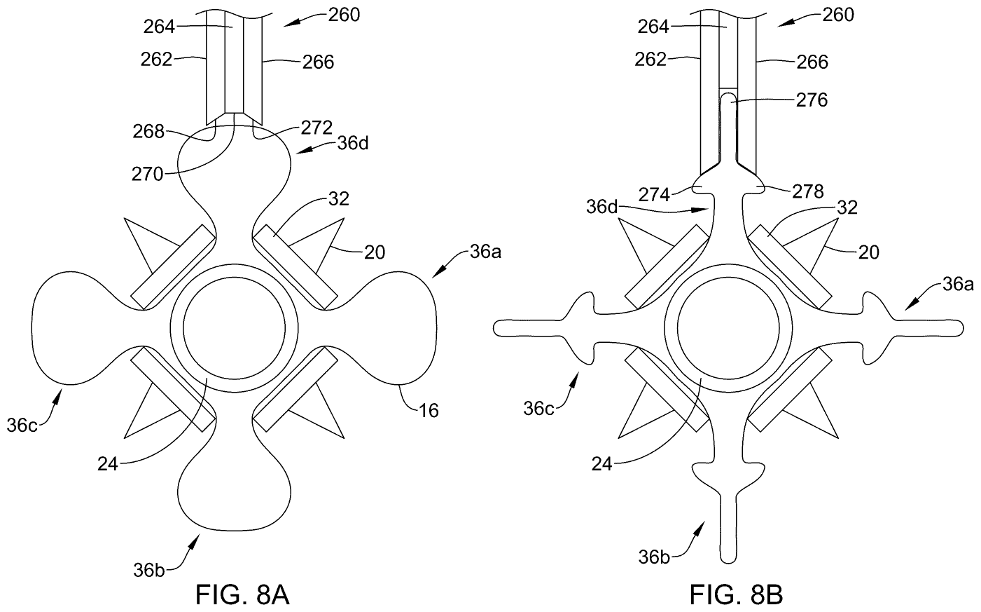

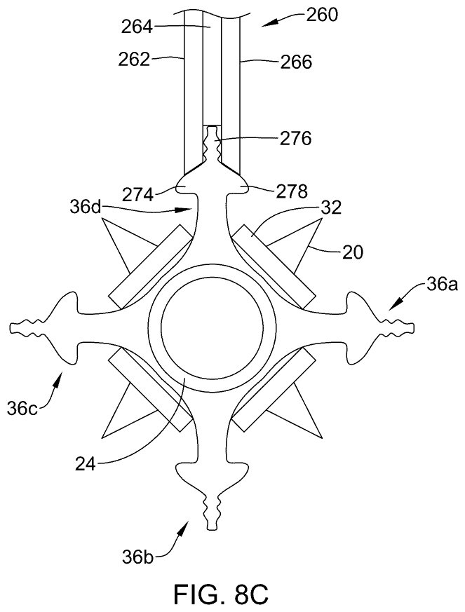

FIGS. 8A-8C are schematic partial cross-sectional views of a balloon folding process having an alternative balloon pusher element 260 that may be used in the balloon folding tool 100. While a single balloon pusher element 260 is shown in FIGS. 8A-8C, it should be understood that a plurality of balloon pusher elements 260 may be utilized. For example, a balloon pusher element 260 may be provided for each wing 36. Further, the balloon pusher elements 260 may each be actuated in a similar manner to form wings 36 having similar shapes. The balloon pusher element 260 may be used in the balloon folding tool 100 in place of one or more of the balloon pusher elements 106.

The balloon pusher element 260 may include a first balloon pusher member 262, a second balloon pusher member 264, and a third balloon pusher member 266. The first, second, and third balloon pusher members 262, 264, 266 may be individually actuatable. For example, the first balloon pusher member 262 may be movable independent of the second balloon pusher member 264 and the third balloon pusher member 266, the second balloon pusher member 264 may be movable independent of the first balloon pusher member 262 and the third balloon pusher member 266, and the third balloon pusher member 266 may be movable independent of the first balloon pusher member 262 and the second balloon pusher member 264. Each of the first, second, and third balloon pusher members 262, 264, 266 extend from a radially outward end (not explicitly shown) to a radially inward end 268, 270, 272. The first and third radially inward ends 268, 272 may extend at non-orthogonal angles relative to a longitudinal axis of the first and third balloon pusher members 262, 266 while the second radially inward end 270 may extend at a generally orthogonal angle relative to a longitudinal axis of the second balloon pusher member 264. In some examples, the first and third radially inward ends 268, 272 may be mirror images of one another, or may extend at opposite angles to one another. The first, second, and third radially inward ends 268, 270, 272 may be generally linear. However, this is not required. The radially inward ends 268, 270, 272 may take other shapes and/or configurations as desired. Other shapes of the radially inward ends 268, 270, 272 may include, but are not limited to, pear shaped, rounded, concave, convex, undulating, or the like. It is contemplated that the shape of the radially inward ends 268, 270, 272 may impact the final shape of the wings 36. The radially inward ends 268, 270, 272 may have similar shapes or differing shapes, as desired. It is further contemplated that the first, second, and third balloon pusher members 262, 264, 266 may have similar widths or differing widths, as desired.

In FIGS. 8A-8C, the portions of the balloon 16 adjacent to the blades 20 have already been folded. For brevity and case of understanding, the blade pusher elements (e.g., similar in form and function to blade pusher elements 104 described herein) are not shown. However, it should be understood that the balloon pusher elements 260 may be used in combination with blade pusher elements. FIG. 8A illustrates the balloon folding process just after the blade pusher elements have been actuated. In FIG. 8B, the first balloon pusher member 262 and the third balloon pusher member 266 of the balloon pusher element 260 have been advanced radially inwards while the second balloon pusher member 264 remains stationary. The first balloon pusher member 262 may push or displace a first portion 274 of the balloon 16 adjacent thereto to reduce the outer profile of the balloon 16 and the third balloon pusher member 266 may push or displace a third portion 278 of the balloon 16 adjacent thereto to reduce the outer profile of the balloon 16. In some examples, the first and third balloon portions 274, 278 may have sloped surfaces generally conforming to the first and third radially inwards ends 268, 272. However, this is not required. Next, as shown in FIG. 8C, the second balloon pusher member 264 may push or displace a second portion 276 of the balloon 16 adjacent thereto to reduce the outer profile of a second portion 276 of the balloon 16. In some examples, the second balloon pusher member 264 may be actuated while the first and third balloon pusher members 262, 266 remain in an advanced radially inwards configuration (e.g., to hold the first portion 274 and third portion 278 of the balloon 16 in the folded configuration). In other examples, the first balloon pusher member 262 and/or the third balloon pusher member 266 may be partially or fully radially retracted (e.g., moved radially outwards) before the second balloon pusher member 264 is moved radially inwards or while the second balloon pusher member 264 is being moved radially inwards. In some examples, the second balloon pusher member 264 may fold the second portion 276 on itself to form a wing 36 having a baffled central portion 276 and laterally extending side portions 274, 278. However, it is contemplated that the order or sequence of actuation of the first, second, and third balloon pusher members 262, 264, 266 may be manipulated to change a shape of the wing 36.

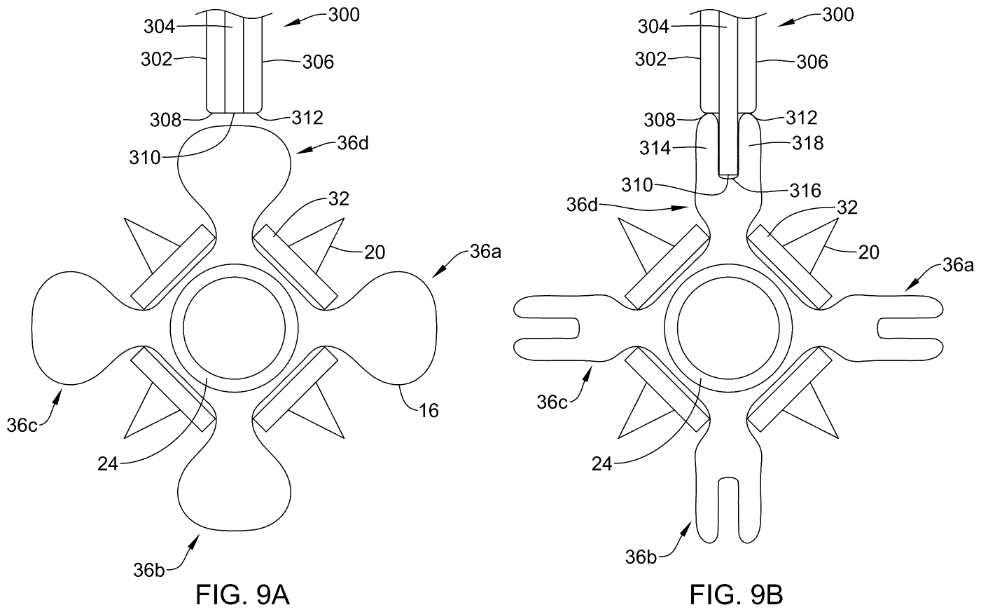

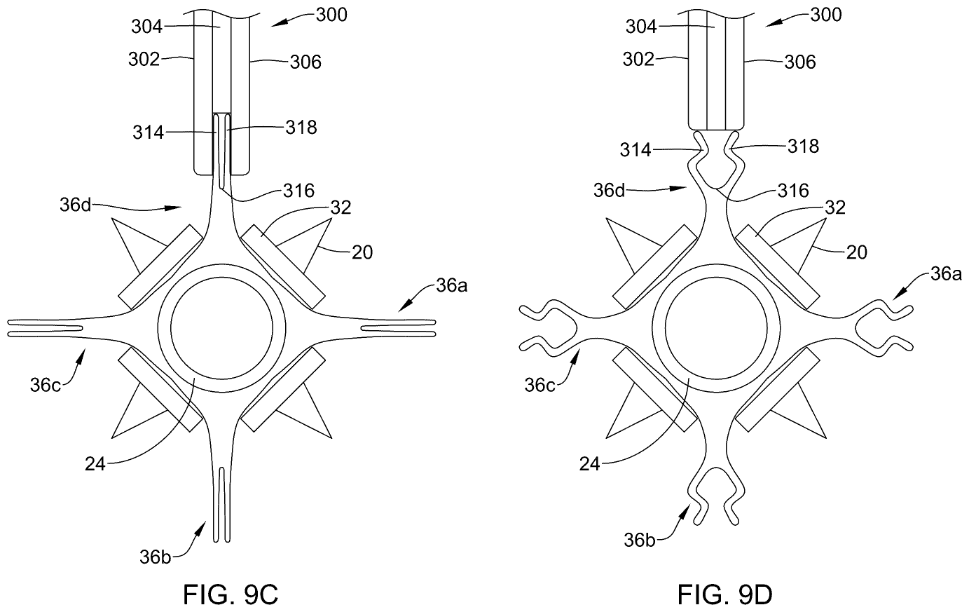

FIGS. 9A-9D are schematic partial cross-sectional views of a balloon folding process having an alternative balloon pusher element 300 that may be used in the balloon folding tool 100. While a single balloon pusher element 300 is shown in FIGS. 9A-9D, it should be understood that a plurality of balloon pusher elements 300 may be utilized. For example, a balloon pusher element 300 may be provided for each wing 36. Further, the balloon pusher elements 300 may each be actuated in a similar manner to form wings 36 having similar shapes. The balloon pusher element 300 may be used in the balloon folding tool 100 in place of one or more of the balloon pusher elements 106.

The balloon pusher element 300 may include a first balloon pusher member 302, a second balloon pusher member 304, and a third balloon pusher member 306. The first, second, and third balloon pusher members 302, 304, 306 may be individually actuatable. For example, the first balloon pusher member 302 may be movable independent of the second balloon pusher member 304 and the third balloon pusher member 306, the second balloon pusher member 304 may be movable independent of the first balloon pusher member 302 and the third balloon pusher member 306, and the third balloon pusher member 306 may be movable independent of the first balloon pusher member 302 and the second balloon pusher member 304. Each of the first, second, and third balloon pusher members 302, 304, 306 extend from a radially outward end (not explicitly shown) to a radially inward end 308, 310, 312. The first, second, and third radially inward ends 308, 310, 312 may extend at generally orthogonal angles relative to a longitudinal axis of the first, second, and third balloon pusher members 302, 304, 306. The first, second, and third radially inward ends 308, 310, 312 may be generally linear. However, this is not required. The radially inward ends 308, 310 may take other shapes and/or configurations as desired. Other shapes of the radially inward ends 308, 310 may include, but are not limited to, pear shaped, rounded, concave, convex, undulating, or the like. It is contemplated that the shape of the radially inward ends 308, 310, 312 may impact the final shape of the wings 36. While the radially inward ends 308, 310, 312 are shown and described as being of a similar shape, the radially inward ends 308, 310, 312 may have differing shapes, as desired. It is further contemplated that the first, second, and third balloon pusher members 302, 304, 306 may have similar widths or differing widths, as desired.

In FIGS. 9A-9D, the portions of the balloon 16 adjacent to the blades 20 have already been folded. For brevity and ease of understanding, the blade pusher elements (e.g., similar in form and function to blade pusher elements 104 described herein) are not shown. However, it should be understood that the balloon pusher elements 300 may be used in combination with blade pusher elements. FIG. 9A illustrates the balloon folding process just after the blade pusher elements have been actuated. In FIG. 9B, the second balloon pusher member 304 of the balloon pusher element 300 has been advanced radially inwards while the first balloon pusher member 302 and the third balloon pusher member 306 remain stationary. The second balloon pusher member 304 may push or displace a second portion 316 of the balloon 16 adjacent thereto to reduce the outer profile of the balloon 16 to form a generally “U” shaped wing 36d with the first balloon portion 314 and the third balloon portion 318 forming laterally spaced legs. Next, as shown in FIG. 9C, the first and third balloon pusher members 302, 306 may be actuated to pinch the first and third portions 314, 318 of the balloon 16 closer together. In some examples, the first and third balloon pusher members 302, 306 may be actuated while the second balloon pusher member 304 remains in an advanced radially inwards configuration (e.g., to hold the second portion 316 of the balloon 16 in the folded configuration). In other examples, the second balloon pusher member 304 may be partially or fully radially retracted (e.g., moved radially outwards) before the first and/or third balloon pushers 302, 306 are moved radially inwards or while the first and/or third balloon pushers 302, 306 are being moved radially inwards. Next, as shown in FIG. 9D each of the three balloon pusher members 302, 304, 306 may be moved radially outwards and subsequently moved radially inwards together to form a double folded wing 36. For example, the first balloon portion 314 may fold upon itself and the third balloon portion 318 may fold upon itself such that each wing 36 includes two folded balloon portions 314, 318. However, it is contemplated that the order or sequence of actuation of the first, second, and third balloon pusher members 302, 304, 306 may be manipulated to change a shape of the wing 36.

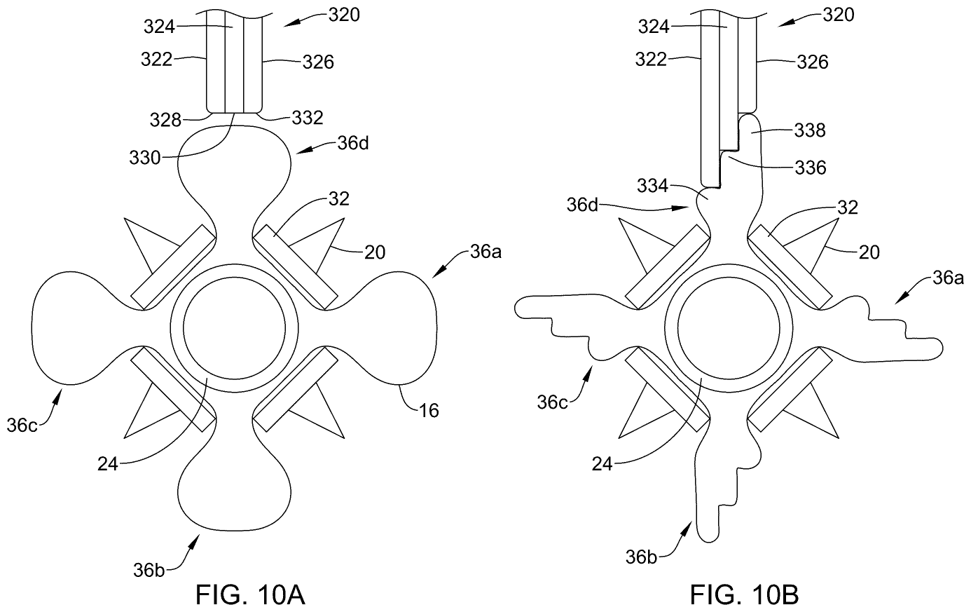

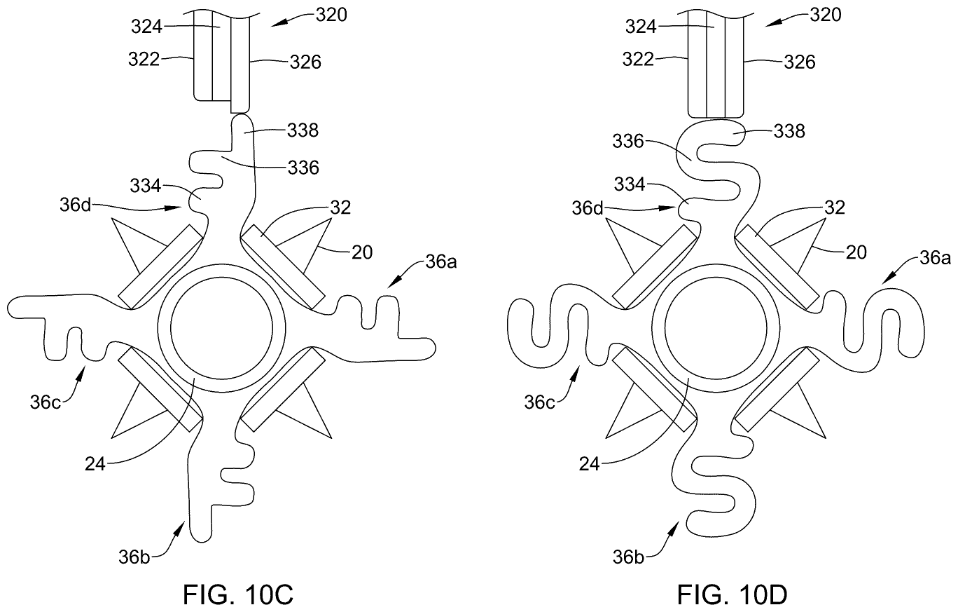

FIGS. 10A-10D are schematic partial cross-sectional views of a balloon folding process having an alternative balloon pusher element 320 that may be used in the balloon folding tool 100. While a single balloon pusher element 320 is shown in FIGS. 10A-10D, it should be understood that a plurality of balloon pusher elements 320 may be utilized. For example, a balloon pusher element 320 may be provided for each wing 36. Further, the balloon pusher elements 320 may each be actuated in a similar manner to form wings 36 having similar shapes. The balloon pusher element 320 may be used in the balloon folding tool 100 in place of one or more of the balloon pusher elements 106.

The balloon pusher element 320 may include a first balloon pusher member 322, a second balloon pusher member 324, and a third balloon pusher member 326. The first, second, and third balloon pusher members 322, 324, 326 may be individually actuatable. For example, the first balloon pusher member 322 may be movable independent of the second balloon pusher member 324 and the third balloon pusher member 326, the second balloon pusher member 324 may be movable independent of the first balloon pusher member 322 and the third balloon pusher member 326, and the third balloon pusher member 326 may be movable independent of the first balloon pusher member 322 and the second balloon pusher member 324. Each of the first, second, and third balloon pusher members 322, 324, 326 extend from a radially outward end (not explicitly shown) to a radially inward end 328, 330, 332. The first, second, and third radially inward ends 328, 330, 332 may extend at generally orthogonal angles relative to a longitudinal axis of the first, second, and third balloon pusher members 322, 324, 326. The first, second, and third radially inward ends 328, 330, 332 may be generally linear. However, this is not required. The radially inward ends 328, 330, 332 may take other shapes and/or configurations as desired. Other shapes of the radially inward ends 328, 330, 332 may include, but are not limited to, pear shaped, rounded, concave, convex, undulating, or the like. It is contemplated that the shape of the radially inward ends 328, 330, 332 may impact the final shape of the wings 36. While the radially inward ends 328, 330, 332 are shown and described as being of a similar shape, the radially inward ends 328, 330, 332 may have differing shapes, as desired. It is further contemplated that the first, second, and third balloon pusher members 322, 324, 326 may have similar widths or differing widths, as desired.

In FIGS. 10A-10D, the portions of the balloon 16 adjacent to the blades 20 have already been folded. For brevity and case of understanding, the blade pusher elements (e.g., similar in form and function to blade pusher elements 104 described herein) are not shown. However, it should be understood that the balloon pusher elements 320 may be used in combination with blade pusher elements. FIG. 10A illustrates the balloon folding process just after the blade pusher elements have been actuated. In FIG. 10B, the first balloon pusher member 322 and the second balloon pusher member 324 of the balloon pusher element 320 may be separately or independently advanced radially inwards (e.g., simultaneously or sequentially) while the third balloon pusher member 324 remains stationary. For example, in a sequential operation, the first balloon pusher member 322 may push or displace a first portion 334 of the balloon 16 adjacent thereto to reduce the outer profile of the balloon 16. Next, the second balloon pusher member 324 may be advanced radially inwards while the first balloon pusher member 322 and the third balloon pusher member 326 remain stationary. The second balloon pusher member 324 may push or displace a second portion 336 of the balloon 16 adjacent thereto to reduce the outer profile of the balloon 16. In some cases, the first balloon pusher member 322 may be partially or completely moved radially outwards prior to actuating the second balloon pusher member 324. However, this is not required. Next, as shown in FIG. 10C, the third balloon pusher member 326 may be advanced radially inwards. The third balloon pusher member 326 may push or displace a third portion 338 of the balloon 16 adjacent thereto to reduce the outer profile of the balloon 16. In some cases, the first and/or second balloon pusher members 322, 324 may be partially or completely moved radially outwards and held stationary prior to actuating the third balloon pusher member 326. However, this is not required. In other examples, the first and/or second balloon pusher members 322, 324 may remain stationary in a radially advanced configuration. Next, as shown in FIG. 10D each of the three balloon pusher members 322, 324, 326 may be moved radially outwards and subsequently moved radially inwards together to form an undulating or “S” shaped wing 36. However, it is contemplated that the order or sequence of actuation of the first, second, and third balloon pusher members 322, 324, 326 may be manipulated to change a shape of the wing 36.

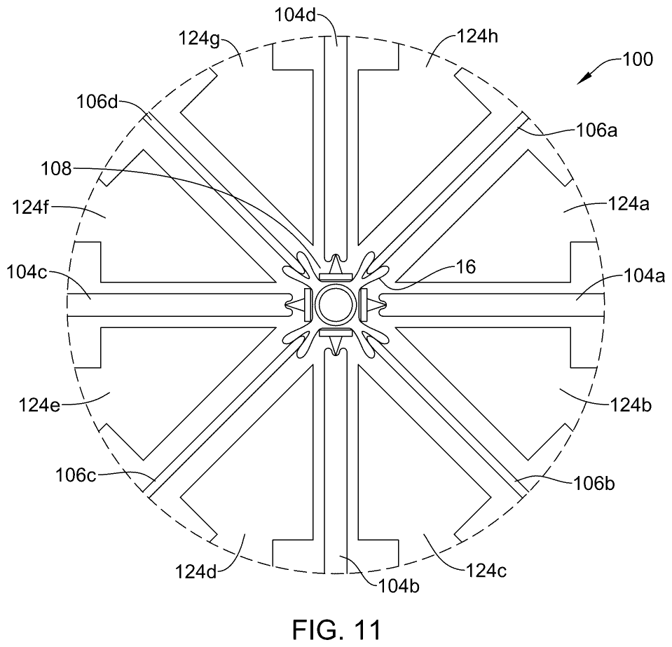

In some embodiments, additional components of the balloon folding tool 100 may be configured to be actuatable to utilize components thereof to facilitate folding of the wings 36 with multiple elements folding each wing 36. FIG. 11 is an enlarged partial cross-sectional view of the balloon folding tool 100 with the balloon 16 disposed within the central aperture 108. For brevity and case of understanding, every feature has not been provided with a reference number. The housing 102 may include wedge-shaped members 124a-h (collectively, 124) positioned between an adjacent blade pusher element 104 and balloon pusher element 106. Said differently each blade pusher element 104 and each balloon pusher element 106 may have a wedge-shaped member 124 on either side thereof. It is contemplated that the wedge-shaped members 124 may take any shape desired. While not explicitly shown, the balloon folding tool 100 may further include one or more actuation mechanisms movably coupled to the wedge-shaped members 124. The actuation mechanisms may be configured to radially displace the wedge-shaped members 124. Each balloon pusher element 106 may be actuated with one or both of the wedge-shaped members 124 positioned on either side thereof in the manners described with respect to FIGS. 5A-5C, 6A-6C, 7A-7C, 9A-9D, or 10A-10D to use multiple elements to fold each wing 36 of the balloon 16.

It is further contemplated that the pressure of the balloon 16 may be manipulated during the folding process to facilitate folding and/or to manipulate the shape of the folded balloon 16. In one example, the balloon 16 may be inflated to a first pressure. In an illustrative example, the first pressure may be 15 pounds per square inch (psi) (103.4 kilopascal). The blade pusher elements 104 may be actuated with the balloon 16 at the first pressure. Next the pressure may be reduced and a pusher element actuated to form an intermediary shape. Finally, a vacuum may be applied and one or more pusher elements actuated to form folds in the intermediary shape. The balloon 16 may be heat set in the final folded form. In some embodiments, a higher pressure (e.g., in the range of about 100 psi (689 kilopascal) may be applied to the folded balloon 16 during the heat set to force the shape into the full folded balloon 16.

In another example, the balloon 16 may be inflated to a first pressure. In an illustrative example, the first pressure may be 15 psi (103.4 kilopascal). The blade pusher elements 104 may be actuated with the balloon 16 at the first pressure. Next the pressure may be reduced and a pusher element actuated to form an intermediary shape. A vacuum may be applied to the balloon 16 and the balloon 16 heat set into the intermediary shape. Next, one or more pusher elements may be actuated to push the heat set wings 36 towards the center of the balloon 16.

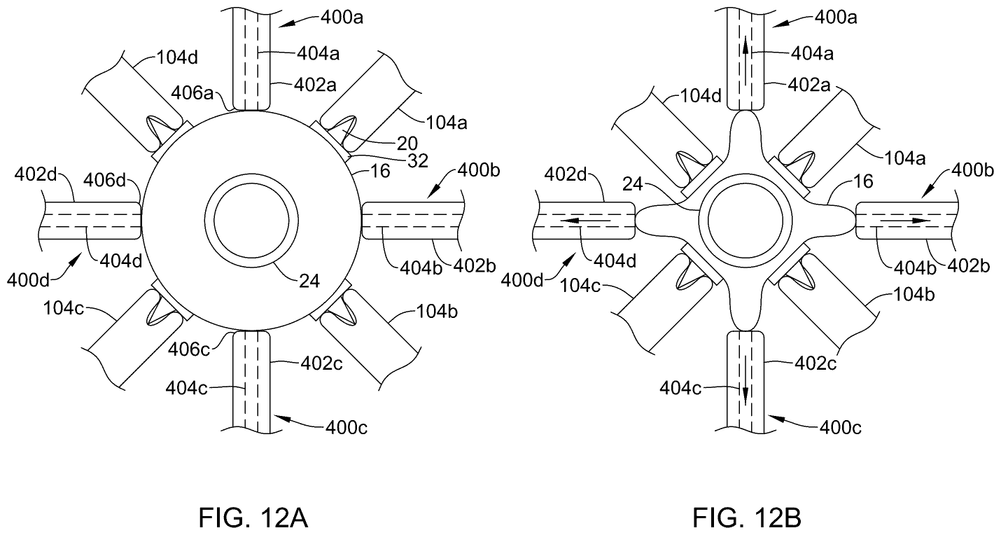

In some embodiments, a vacuum may be used to fold and/or manipulate the balloon wings 36 during the folding process. FIGS. 12A-12B are schematic partial cross-sectional views of a balloon folding process having an alternative balloon pusher element 400a, 400b, 400c, 400d (collectively, 400) that may be used in the balloon folding tool 100. The balloon pusher element 400 may be used in the balloon folding tool 100 in place of one or more of the balloon pusher elements 106. Each balloon pusher element 400 may include an elongate shaft 402a, 402b, 402c, 402d (collectively, 402) having a lumen 404a, 404b, 404c, 404d (collectively, 404) extending therethrough. The lumens 404 may be in fluid communication with a vacuum source. In some examples, the pusher element 400 may be formed from two laterally spaced components with the space between the two components defining the lumen. To fold the balloon 16, the blade pusher members 104 may be actuated (displaced radially inwards) to partially fold the balloon 16, as shown in FIG. 12B. The blade pusher members 104 may be actuated with the balloon 16 at a first pressure. Once the blade pusher members 104 have been actuated, the pressure within the balloon 16 may be reduced. Prior to actuating the blade pusher members 104, a vacuum may be pulled through the lumens 404 of the balloon pusher members 400. The radially inward end 406a, 406b, 406c, 406d (collectively, 406) of the balloon pusher members 400 may be in contact with the outer surface of the balloon 16 such that as the vacuum is drawn, the balloon 16 is sucked into/towards the lumen 404. This may hold the outer surface of the balloon 16 against the balloon pusher member 400. After the blade pusher members 104 have been actuated, the balloon pusher members 400 may be actuated (displaced radially inwards) to complete folding of the balloon 16. While FIGS. 12A and 12B illustrate the balloon pusher members 400 as having a single element, the balloon pusher members may have more than one element, as desired.

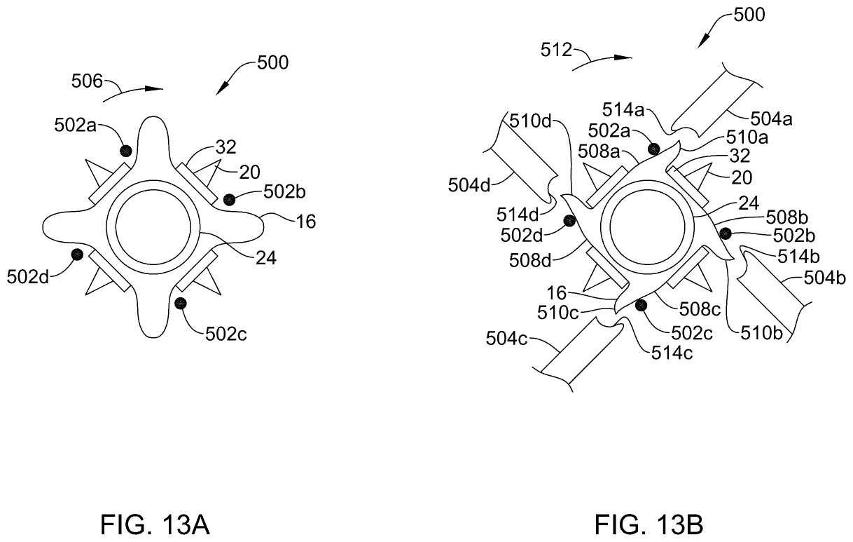

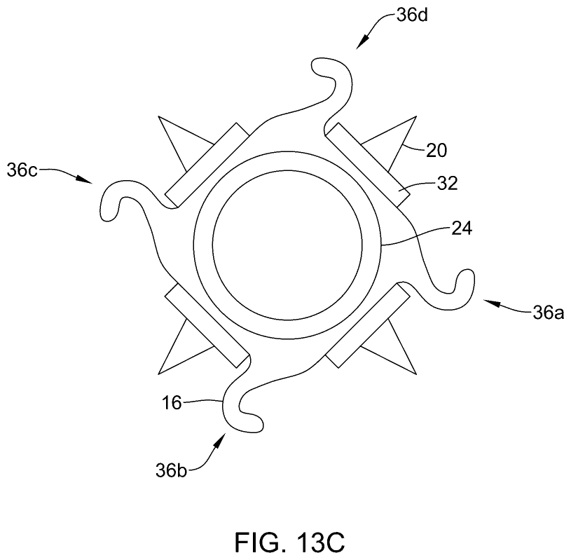

FIGS. 13A-13C are schematic partial cross-sectional views of a balloon folding process having an alternative balloon pusher element system 500 that may be used in the balloon folding tool 100. The balloon pusher element system 500 may be used in the balloon folding tool 100 in place of one or more of the balloon pusher elements 106. In FIGS. 13A-13C, the portions of the balloon 16 adjacent to the blades 20 have already been folded. For brevity and ease of understanding, the blade pusher elements (e.g., similar in form and function to blade pusher elements 104 described herein) are not shown. However, it should be understood that the balloon pusher element system 500 may be used in combination with blade pusher elements. The balloon pusher element system 500 may include a plurality of bars or rods 502a, 502b, 502c, 502d (collectively, 502) and a plurality of balloon pusher elements 504a, 504b, 504c, 504d (collectively, 504). The rods 502 may be circumferentially spaced within the central aperture and extend along a length of the housing 102 or a length of balloon 16. Generally, the rods 502 may be configured to rotate about a longitudinal axis of the central aperture 108 in a first direction to form a first portion of the folding process. In an initial configuration, the rods 502 may be disposed adjacent to a side of the mounting pad 32. In some examples, the actuation of the blade pusher elements may move the rods 502 radially inward until they contact an outer surface of the balloon adjacent to a side of the mounting pads 32. The rods 502 may then be rotated in a first, or clockwise, direction, as shown at arrow 506 in FIG. 13A to partially compress a first portion 508a, 508b, 508c, 508d (collectively, 508) the balloon 16, as shown in FIG. 13B. As the rods 502 are rotated, the wings 36 may be biased in the direction of movement of the rods 502. Depending on the placement of the rods with respect to the cutting blades 20, in some cases, the rods 502 may be rotated in a counterclockwise direction.

Next, the balloon pusher elements 504 may be actuated (e.g., moved radially inwards) to contact a second portion 510a, 510b, 510c, 510d (collectively, 510). The balloon pusher elements 504 may engage the second portion 510 of the wings 36. As the balloon pusher elements 504 are actuated, the balloon pusher elements 504 may be rotated about the longitudinal axis of the central aperture 108 in a second direction, opposite the first direction, as shown at arrow 512 in FIG. 13B. As the balloon pusher elements 504 are rotated, the wings 36 may be biased in the direction of movement of the balloon pusher elements 504 to form a wing 36 which curls back on itself, as shown in FIG. 13C.

The balloon pusher elements 504 extend from a radially outward end (not explicitly shown) to a radially inward end 514a, 514b, 514c, 514d (collectively, 514). The radially inward ends 514 may be generally concave. In some examples, the concave shape may be asymmetrical. However, this is not required. The radially inward ends 514 may take other shapes. Other shapes of the radially inward ends 514 may include, but are not limited to, pear shaped, rounded, linear (extending at orthogonal or non-orthogonal angles relative to a longitudinal axis of the balloon pusher element 504), convex, undulating, or the like. It is contemplated that the shape of the radially inward ends 514 may impact the final shape of the wings 36. While the radially inward ends 514 are shown and described as being of the same shape, the radially inward ends 514 may have differing shapes, as desired.

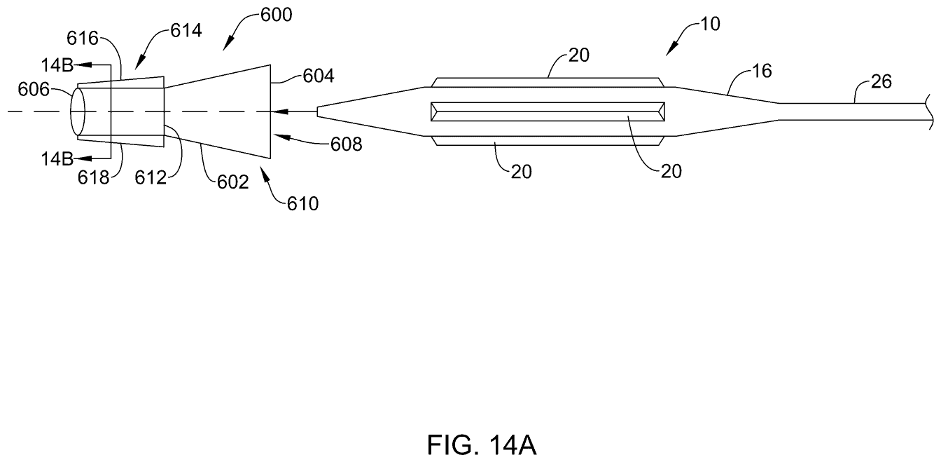

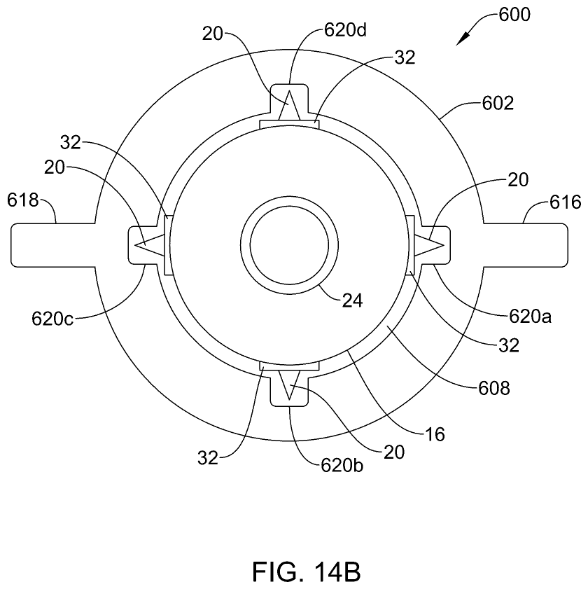

In some cases, the balloon 16 may be loaded into the balloon folding tool 100 with the assistance of an alignment and loading fixture. FIG. 14A is a side view of an illustrative balloon catheter 10 with an alignment and loading fixture 600. The alignment and loading fixture 600 may include a housing 602 extending from a first end 604 to a second end 606. The housing 602 defines a lumen 608 extending from the first end 604 to the second end 606. The housing 602 may include a first end region 610 extending from the first end 604 to an intermediate location 612. The first end region 610 may have an outer diameter that tapers or reduces from the first end 604 to the intermediate location 612. The inner diameter of the first end region 610 may also taper from the first end 604 to the intermediate location 612. The housing 602 may further include a second end region 614 extending from the intermediate location 612 to the second end 606. The second end region 614 may have an outer diameter that remains substantially constant or uniform from the intermediate location 612 to the second end 606. The inner diameter of the second end region 614 may also be substantially constant or uniform from the intermediate location 612 to the second end 606.

A first guide 616 may extend laterally from a first side of the second end region 614 and a second guide 618 may extend laterally from a second side of the second end region 614. In some embodiments, the first guide 616 and the second guide 618 may be spaced approximately 180° from one another. However, other spacings may be used, as desired. Further, the alignment and loading fixture 600 may include fewer than two or more than two guides 616, 618, as desired. The guides 616, 618 may be configured to be received within mating recesses within the balloon folding tool 100 (not explicitly shown in FIG. 14A). When the guides 616, 618 are assembled with the mating recesses within the balloon folding tool 100 and the balloon 16 is advanced through the alignment and loading fixture 600 and into the balloon folding tool 100, the blades 20 of the balloon 16 may align with the blade pusher elements 104 of the balloon folding tool 100.

Referring additionally to FIG. 14B which is a cross-sectional view of the alignment and loading fixture 600 taken at line 14B-14B of FIG. 14A with a balloon 16 disposed therein, the alignment and loading fixture 600 may include one or more channels 620a, 620b, 620c, 620d (collectively, 620). The channels 620 may extend radially outward from the lumen 608 to increase a cross-sectional dimension of the lumen 608 at the channels 620. The channels 620 may have a size and shape configured to receive the blades 20 of the balloon 16 without damaging the blades 20. The channels 620 may extend from the first end 604 to the second end 606 of the housing 602. The alignment and loading fixture 600 may include a same number of channels 620 as there are cutting blades 20 on the balloon 16. Thus, the alignment and loading fixture 600 may include fewer than four or more than four channels 620. Further, the channels 620 may be arranged to align with the cutting blades 20. In some cases, visual indicia may be provided on an outer surface of the housing 602 to help the user or machinery align the cutting blades 20 with the channels 620. It is contemplated that the funnel shape of the first end region 610 may help to guide the blades 20 into the channels 620. Further, the inner diameter of the lumen 608 adjacent the first end 604 may be greater than the outer diameter of the balloon 16 at the cutting blades 20 to facilitate insertion thereof. The inner diameter of the lumen 608 within the second end region 614 of the housing 602 may be less than the outer diameter of the balloon 16 at the cutting blades 20 such that the cutting blades 20 are directed into the channels 620. Said differently, the inner diameter of the lumen 608 within the second end region 614 of the housing 602 may be sized such that the balloon 16 will not advance unless the cutting blades 20 are disposed within the channels 620.

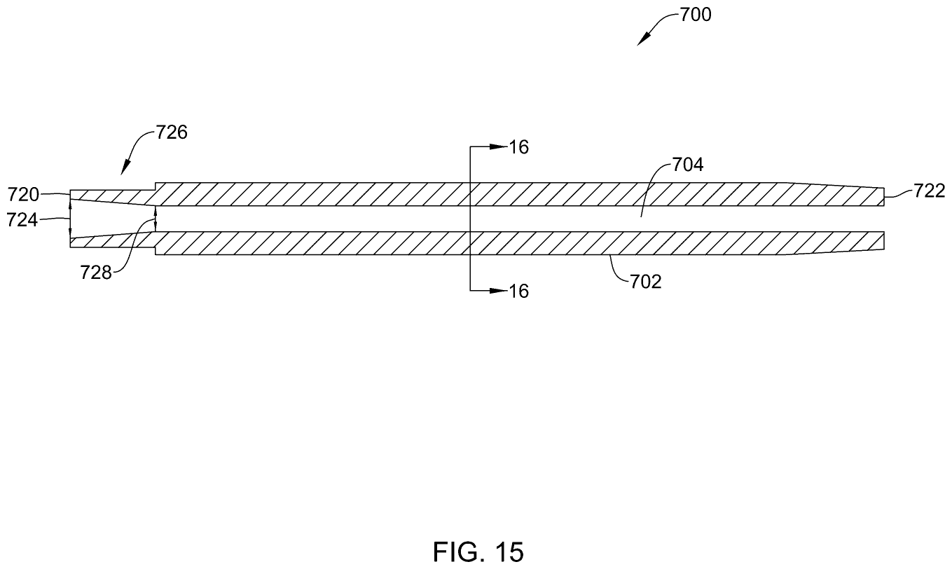

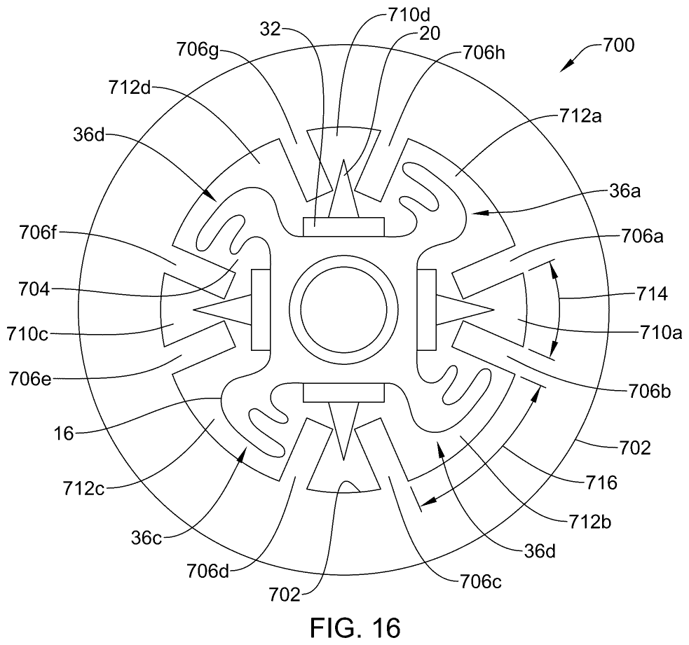

Once the balloon 16 has been folded, it may be desirable to position the balloon 16 within a balloon protector to maintain the balloon 16 in the folded configuration and to prevent damage to the balloon 16. FIG. 15 is a schematic cross-sectional view of an illustrative balloon protector 700. The balloon protector 700 may include a housing 702 extending from a first end 720 to a second end 722. The length of the housing 702 may be sufficient to receive an entirety of a length of the balloon 16 therein. In some embodiments, the housing 702 may be a generally tubular member defining a lumen 704 extending along the length thereof (e.g., from the first end 722 to the second end 724). A first end region 726 of the housing 702 may have a tapered inner diameter. For example, the inner diameter may taper from a first inner diameter 724 adjacent the first end 720 thereof to a second, smaller, inner diameter 728. In some examples, the first inner diameter 724 may be in the range of about 0.060 inches to about 0.074 inches (1.524 millimeters to about 1.880 millimeters). In some examples, the second inner diameter 728 may be about 0.041 inches (1.041 millimeters). However, the first and/or second inner diameters 724, 728 may be larger or smaller, as desired. It is contemplated that the tapered or funnel shape of the first lumen 704 adjacent the first end region 726 may facilitate assembly of the balloon 16 with the balloon protector 700 by reducing assembly forces, enabling fold shape progression and/or enabling better proximal cone folding. For example, the funnel shape may make it easier to align the folded balloon with the lumen and facilitate insertion. The proximal waist of the balloon 16 may rest within the funnel region. The increased diameter of the funnel region may facilitate folding of the proximal cone.

FIG. 16 is a schematic cross-sectional view of the illustrative balloon protector 700 of FIG. 15 taken at line 16-16 thereof and having a folded balloon 16 disposed therein. One or more walls 706a-h (collectively, 706) may extend radially inwards into the lumen 704 from an inner surface 708 of the housing 702. A length of the walls 706 may be selected based on the height of the cutting blades 20 and/or the size of the wings 36. The walls 706 may be eccentrically spaced to define a first plurality of recesses 710a, 710b, 710c, 710d (collectively, 710) sized and shaped to receive the cutting blades 20 therein and a second plurality of recesses 712a, 712b, 712c, 712d (collectively, 712) sized and shaped to receive the wings 36 therein. It is contemplated that the second plurality of recesses 712 may be equal to or larger than the first plurality of recesses 710. Said differently, an arc length 714 of the first plurality of recesses 710 may be equal to or less than an arc length 716 of the second plurality of recesses 712. It is contemplated that providing larger recesses 712 for the wings 36 may help limit migration and/or pinching of the wings 36 when the balloon 16 is within the balloon protector 700.

The materials that can be used for the various components of the system(s) and the various elements thereof disclosed herein may include those commonly associated with medical devices. For simplicity purposes, the following discussion refers to the system. However, this is not intended to limit the devices and methods described herein, as the discussion may be applied to other elements, members, components, or devices disclosed herein, such as, but not limited to, the catheter shaft, the inflatable balloon, the cutting members, the emitter shaft, etc., and/or elements or components thereof.

In some embodiments, the system, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), expanded polytetrafluoroethylene (ePTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments components can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system, and/or components or portions thereof, may be made of a material that does not substantially distort the image and create substantial artifacts (i.e., gaps in the image). Certain ferromagnetic materials, for example, may not be suitable because they may create artifacts in an MRI image. The system, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antincoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the present disclosure. This may include, to the extent that it is appropriate, the use of any of the features of one example embodiment being used in other embodiments. The scope of the present disclosure is, of course, defined in the language in which the appended claims are expressed.

What is claimed is:

1. A tool for imparting a plurality of folds to a balloon, the tool comprising:
a housing, the housing defining a central aperture extending along a length of the housing;
a plurality of blade pusher elements spaced about a circumference of the housing, the plurality of blade pusher elements each extending from a radially outward edge to a radially inward portion, the radially inward portion configured to extend into the central aperture; and
a plurality of balloon pusher elements spaced about a circumference of the housing, the plurality of balloon pusher elements each extending from a radially outward edge to a radially inward end, the radially inward end configured to extend into the central aperture;
wherein each balloon pusher element of the plurality of balloon pusher elements comprises two or more balloon pusher members.

2. The tool of claim 1, wherein the plurality of blade pusher elements are configured to be radially displaced.

3. The tool of claim 1, wherein the plurality of balloon pusher elements are configured to be radially displaced.

4. The tool of claim 1, wherein each balloon pusher member of the two or more balloon pusher members are independently actuatable.

5. The tool of claim 1, wherein a radially inward end of at least one of the two or more balloon pusher members is generally linear.

6. The tool of claim 5, wherein the radially inward end of the least one of the two or more balloon pusher members extends generally orthogonal to a longitudinal axis of the at least one balloon pusher member.

7. The tool of claim 5, wherein the radially inward end of the least one of the two or more balloon pusher members extends at a non-orthogonal angle to a longitudinal axis of the at least one balloon pusher member.

8. The tool of claim 1, wherein a radially inward end of at least one of the two or more balloon pusher members is generally concave.

9. The tool of claim 1, wherein a radially inward end of at least one of the two or more balloon pusher members is generally convex.

10. The tool of claim 1, wherein each balloon pusher element of the plurality of balloon pusher elements comprises three or more balloon pusher members.

11. The tool of claim 1, further comprising a lumen extending through each balloon pusher member of the plurality of balloon pusher members.

12. The tool of claim 1, wherein the radially inward portion of each blade pusher element of the plurality of pusher elements defines a channel.

13. A tool for imparting a plurality of folds to a balloon, the tool comprising:
a housing, the housing defining a central aperture extending along a length of the housing;
a plurality of blade pusher elements spaced about a circumference of the housing, the plurality of blade pusher elements each extending from a radially outward edge to a radially inward portion, the radially inward portion configured to extend into the central aperture;
a plurality of balloon pusher elements spaced about a circumference of the housing, the plurality of balloon pusher elements each extending from a radially outward edge to a radially inward end, the radially inward end configured to extend into the central aperture; and
a plurality of rods circumferentially spaced within the central aperture and extending along a length of the housing.

14. The tool of claim 13, wherein the plurality of rods are configured to be rotated about a longitudinal axis of the central aperture in a first direction.

15. The tool of claim 14, wherein the balloon pusher elements are configured to be rotated about the longitudinal axis of the central aperture in a second direction opposite the first direction.

16. The tool of claim 13, wherein the radially inward end of at least one of the plurality of balloon pusher elements is generally convex.

17. A method for folding a balloon, the method comprising:
inserting a balloon into a housing of a balloon folding tool;
moving a plurality of blade pusher elements radially inwards to compress a first portion of the balloon; and
sequentially moving two or more circumferentially adjacent balloon pusher members radially inwards to compress a second portion of the balloon and form a folded portion.

18. The method of claim 17, wherein moving the plurality of blade pusher elements radially inwards is performed at a first pressure.

19. The method of claim 18, wherein sequentially moving two or more circumferentially adjacent balloon pusher members radially inwards is performed at a second pressure less than the first pressure.

20. The method of claim 17, after sequentially moving two or more circumferentially adjacent balloon pusher members, heat setting the balloon.
